(19) United States
(12) Patent Application Publication
(10) Pub. No.: US 2011/0281945 A1
(43) Pub. Date: Nov. 17, 2011

(54) GASTRIC ACID SECRETION INHIBITOR, AND POTASSIUM CHANNEL INHIBITOR

(76) Inventors: Yuichi Suzuki, Shizuoka (JP); Kenya Ishida, Kanagawa (JP); Mitsuhiko Fujiwhara, Kanagawa (JP)

(21) Appl. No.: 13/146,250

(22) PCT Filed: Jan. 26, 2010
(86) PCT No.: PCT/JP10/00424

§ 371 Date: Jul. 26, 2011

(30) Foreign Application Priority Data
Jan. 27, 2009 (JP) 2009-015757

(51) Int. Cl. A61K 31/22; A61K 31/075; C07C 35/12; C07C 233/58; C07C 69/675; C07C 69/68; C07C 35/08; C07C 233/05; C07C 49/407; A61K 31/045; A61K 31/164; A61K 31/122; A61P 1/04; A61P 9/06; C07C 43/196

(52) U.S. Cl. 514/546; 568/670; 514/715; 568/829; 564/123; 560/188; 568/834; 564/215; 568/376; 514/724; 514/739; 514/613; 514/629; 514/690

(57) ABSTRACT

To provide a drug, quasi-drug, food or drink that can act as a gastric acid secretion suppressant or a potassium channel inhibitor by including a composition that can be safely used and can be inexpensively produced. Each of these products contains one or more cooling sensation compositions that are compounds providing cooling sensation in an amount sufficient for exhibiting the efficacy thereof in a living body to which the product is administered.

### TECHNICAL FIELD

The present invention relates to a gastric acid secretion suppressant and a potassium channel inhibitor and relates to a drug, quasi-drug, food or drink containing the suppressant or the inhibitor.

### BACKGROUND ART

Potassium channels are known as a very diverse family and play very important roles in various biological activities. Inhibitors of the potassium channels are promising compounds as various drugs. For example, they are effective to arrhythmia, angina pectoris, peptic esophagitis, motility disturbance (including constipation and diarrhea), gastrointestinal disorder (including irritable bowel syndrome), asthma, and hyperglycemia. Accordingly, a large number of potassium channel inhibitors have been developed and have been used as drugs.

For example, Patent Literature 1 describes a compound having a cyclohexane ring as a potassium channel inhibitor that is effective against a large number of syndromes including the above-mentioned diseases. However, in development of an inhibitor, it is usually necessary to design a very complicated molecule, causing a disadvantage in the purpose of providing an inexpensive product.

On the other hand, recently, use of herbs has been receiving attention as an alternative therapy, and natural mint has been known to have various biological activities.

Natural mint is widely used as food and easily available and also safely contributes to health. For example, menthol and menthone, which are main ingredients of mint, play an important part in intestinal regulation.

The present inventors have disclosed Patent Literatures 2 and 3 in connection with this.

It is believed that suppression of colonic Cl⁻ secretion is highly involved in the mechanisms of action of menthol and menthone. That is, intestinal juice secretion (Cl⁻ secretion) in an epithelial cell of the intestine is, as shown in FIG. 1, caused by taking Na⁺, K⁺, and Cl⁻ into the inside of the cell from the serosa side through a Na.K.2Cl cotransporter on the serosa side, but the Cl⁻ secretion does not smoothly proceed if circulation of K⁺ does not occur.

As one of major K⁺ channels that play a role in this circulation, KCNQ1 (another name: Kv7.1, KVLQT1) is known. It is known that chromanol 293B inhibits the KCNQ1 K⁺ channel to suppress Cl⁻ secretion, and experiments have shown that cooling sensation materials suppress Cl⁻ secretion by the same mechanism as that of chromanol 293B.

This suggests that cooling sensation materials have effects of inhibiting the KCNQ1 K⁺ channel.

Since it is known that the KCNQ1 K⁺ channel plays an important role in several tissues, the cooling sensation materials such as menthol and menthone are expected to widely promote good health.

However, not only menthol and menthone but also their analogs and derivatives and compound groups having similar cooling sensation effects, so-called cooling sensation agents, have many unknown aspects in their biological activities, and their actions on potassium channels have not been elucidated at all.

### PRIOR ART

### Patent Literature

1: Japanese Translation of PCT International Application Publication No. 2006-508016

2: Japanese Patent Application No. 2006-97890

### SUMMARY OF INVENTION

### Technical Problem

Accordingly, it is an object of the present invention to provide a drug, quasi-drug, food or drink that functions as a gastric acid secretion suppressant or a potassium channel inhibitor by containing a composition that can be safely used and also can be inexpensively produced.

### Solution to Problem

In order to solve the above-described problems, the gastric acid secretion suppressant of the present invention has the following constitution. That is, the gastric acid secretion suppressant includes one or more cooling sensation compositions that are compounds providing cooling sensation in an amount sufficient for exhibiting the efficacy thereof in a subject to which the suppressant is administered.

As the cooling sensation composition to be used, at least one selected from menthol, menthone, isopulegol, 3-(menthoxy)propane-1,2-diol, 2-(menthoxy)ethan-1-ol, 2-[2-(menthoxy)ethoxy]ethan-1-ol, 3-(menthoxy)propan-1-ol, 2-methyl-3-(menthoxy)propane-1,2-diol, para-menthane-3,8-diol, menthyl 3-hydroxybutanoate, 1-(2-hydroxy-4-methyl-cyclohexyl)-ethanone, N-ethyl menthyl carboxamide, menthyl lactate, and N-methyl-2,2-isopropylmethyl-3-methylbutanamide is useful.

The cooling sensation composition may have a para-menthane-skeleton and a polar site at the 3-position thereof.

The cooling sensation composition may be provided as a drug, quasi-drug, food or drink produced by mixing the gastric acid secretion suppressant as a main ingredient or an accessory ingredient with another composition.

In the present invention, the cooling sensation composition may be provided as a potassium channel inhibitor by using the cooling sensation composition as an agent inhibiting the potassium channel.

Examples of the agent include a potassium channel inhibitor of which cooling sensation composition has an activity effective for improving arrhythmia; a potassium channel inhibitor of which cooling sensation composition has an activity effective for improving angina pectoris; a potassium channel inhibitor of which cooling sensation composition has an activity effective for improving peptic esophagitis; a potassium channel inhibitor of which cooling sensation composition has an activity effective for improving motility disturbance; a potassium channel inhibitor of which cooling sensation composition has an activity effective for improving gastrointestinal disorder; a potassium channel inhibitor of which cooling sensation composition has an activity effective for improving asthma; and a potassium channel inhibitor of which cooling sensation composition has an activity effective for improving hyperglycemia.

The cooling sensation composition may be provided as a drug, quasi-drug, food or drink produced by mixing the potassium channel inhibitor as a main ingredient or an accessory ingredient with another composition.

### Advantageous Effects of Invention

According to the gastric acid secretion suppressant of the present invention, the action of the cooling sensation composition effectively contributes to suppression of gastric acid secretion without causing adverse reactions. Furthermore, the cooling sensation composition serving as a potassium channel inhibitor effectively contributes to improvement of diseases and symptoms such as arrhythmia, angina pectoris, peptic esophagitis, motility disturbance (including constipation and diarrhea), gastrointestinal disorder (including irritable bowel syndrome), asthma, and hyperglycemia.

### BRIEF DESCRIPTION OF DRAWINGS

FIG. 1 is an explanatory diagram illustrating the mechanism of secretion of intestinal juice (Cl⁻ secretion) in an epithelial cell of the intestine.

FIG. 2 is an explanatory diagram illustrating a configuration for short-circuit current measurement with Ussing chambers.

FIG. 3 is a graph depicting a change in short-circuit current, showing the effects on Cl⁻ secretion when cooling sensation composition, compound 1, and chromanol 293B were administered to the serosa side.

FIG. 4 is a graph depicting a change in short-circuit current in another example.

FIG. 5 is a graph depicting a change in short-circuit current, showing the effects on Cl⁻ secretion when cooling sensation composition, menthol, and chromanol 293B were administered to the serosa side.

FIG. 6 is an explanatory diagram illustrating a method of measuring gastric acid secretion with Ussing chambers.

FIG. 7 includes (a) a pH graph showing the effect by administration of cooling sensation composition, compound 1, and (b) a pH graph showing the effect by administration of cooling sensation composition, 1-menthol.

### DESCRIPTION OF EMBODIMENTS

Embodiments of the present invention will be described below. However, it is possible to appropriately modify the design within the gist of the present invention and to incorporate the technologies described in the patent literatures listed in the Citation List and conventionally known technologies.

There are many unknown aspects in biological activities of cooling sensation agents including menthol, and actions on the potassium channels have not been reported yet.

The present inventors have focused on a compound group having cooling sensation effects as potassium channel inhibitors and have conducted verification experiments to demonstrate their effectiveness on suppression of both intestinal secretion of Cl⁻ and gastric acid secretion in which potassium channels are involved, and reached the present invention based on the findings obtained thereby.

The gastric acid secretion suppressant, the potassium channel inhibitor, and the drug, quasi-drug, food or drink containing the suppressant or the inhibitor of the present invention can further contain various medicinal ingredients as necessary or can be used in a combination with such medicinal ingredients.

The kinds and the total amount of the medicinal ingredients are not particularly limited, and examples of the medicinal ingredients include antacids, stomachics, digestive agents, antiflatulents, other antidiarrhoics, analgesic-antispasmodics, vitamins, amino acids, and other herbal medicines. Examples of further preferred ingredients in the present invention include the following ingredients.

Examples of the antacids include dried aluminum hydroxide gel, magnesium aluminosilicate, magnesium aluminometasilicate, aluminum silicate, hydrotalcite, aluminum magnesium hydroxide, aluminum hydroxide gel, aluminum hydroxide/sodium hydrogen carbonate coprecipitate, aluminum hydroxide/magnesium carbonate co-dried gel, aluminum hydroxide/calcium carbonate/magnesium carbonate coprecipitate, magnesium-based antacids such as magnesium carbonate, magnesium oxide, magnesium hydroxide, magnesium silicate, and magnesium hydroxide/aluminum potassium sulfate coprecipitate, calcium-based antacids such as anhydrous dibasic calcium phosphate, calcium hydrogen phosphate, precipitated calcium carbonate, calcium lactate, and calcium hydroxide, sodium-based antacids such as sodium hydrogen carbonate, sodium citrate, and sodium acetate, anion exchange resins such as polyaminomethylene resins, H2-receptor antagonists such as famotidine, ranitidine, and cimetidine, proton pump inhibitors, and also gastric mucin, cuttlefish bones, abalone, oyster, aminoacetic acid, dihydroxy-aluminum aminoacetate, and scopolia extract.

Examples of the stomachics include herbal medicines such as anise fruit, aloe, fennel, turmeric, Lindera root, plectranthus, Scutellaria root, phellodendron bark, coptis root, processed garlic, zedoary, Pogostemon cablin, cinchona, nux vomica, ginger rhizome, Calamus root, dried ginger, trifoliate orange, immature orange, cinnamon, gentian, red ginseng, magnolia bark, evodia fruit, pepper, calumba, condurango, Zanthoxylum fruit, Hedychium spicatum, sisosi, amomum seed, ginger, cardamon, Citrus reticulata, sweet-flag root, Centaurium minus, swertia herb, atractylodes lancea rhizome, perilla herb, star anise, rhubarb, Panax japonicus rhizome, clove, citrus unshiu peel, capsicum, Picea jezoensis hondoensis, animal bile, picrasma wood, nutmeg, ginseng, mentha herb, Piper longum, atractylodes rhizome, hop, nux vomica extract, buckbean, saussurea root, bitter cardamon, gentian, Alpinia officinarum rhizome, sophora root, sumac gallnut, crataegus fruit, Myrica rubra, mallotus bark, gambir, ubai, cassia seed, and geranium herb; parasympathetic stimulants such as carnitine, neostigmine, bethanechol, carpronium, and tolazoline; antidopaminergic drugs such as metoclopramide, domperidone, and sulpiride; trimebutine; and glutamic acid.

Examples of the digestive agents include starch-digesting enzymes, protein-digesting enzymes, fat-digesting enzymes, cellulose-digesting enzymes, ursodeoxycholic acid, oxycholanic acid hydrochloride, cholic acid, bile powder, bile extract, dehydrocholic acid, and animal bile.

Examples of the enzymes include diastase, pancreatin, pepsin, ptyalin, β-galactosidase, amylase, trypsin, papain, protease, lipase, cellulase, and pancreatin.

Examples of antiflatulents include intestinal bacterial components, gambir, ubai, cassia seed, and geranium herb.

Examples of the other antidiarrhoics include acrinol, berberine chloride, guaiacol, creosote, phenyl salicylate, guaiacol carbonate, berberine tannate, bismuth subsalicylate, bismuth subnitrate, bismuth subcarbonate, bismuth subgallate, tannic acid, albumin tannate, methylenethymoltannin, kaolin, natural aluminum silicate, aluminum hydroxynaphthoate, pectin, medicinal carbon, precipitated calcium carbonate, calcium lactate, calcium hydrogen phosphate, gambir, ubai, phellodendron bark, coptis rhizome, sophora root, geranium herb, sumac gallnut, crataegus fruit, swertia herb, and Myrica rubra.

Examples of the analgesic-antispasmodics include papaverine hydrochloride, ethyl aminobenzoate, scopolamine hydrobromate, scopolamine methylbromide, corydalis tuber, glycyrrhiza, magnolia bark, peony root, timepidium bromide, oxyphencyclimine hydrochloride, dicyclomine hydrochloride, methixene hydrochloride, atropine methylbromide, 1-hyoscyamine methylbromide, methylbenactyzium bromide, belladonna extract, scopolia extract, diphenylpiperidinomethyldioxolan iodide, and total alkaloid citrate of scopolia rhizome.

Examples of the vitamins include vitamin A group, for example, retinal, retinol, retinoic acid, carotene, dehydroretinal, lycopene, and pharmaceutically acceptable salts thereof (e.g., retinol acetate and retinol palmitate); vitamin B group, for example, thiamine, thiamine disulfide, dicethiamine, octotiamine, cycotiamine, bisibutiamine, bisbentiamine, prosultiamine, benfotiamine, fursultiamine, riboflavin, flavin adenine dinucleotide, pyridoxine, pyridoxal, hydroxocobalamin, cyanocobalamin, methylcobalamin, deoxyadenocobalamin, folic acid, tetrahydrofolic acid, dihydrofolic acid, nicotinic acid, nicotinamide, nicotinyl alcohol, pantothenic acid, panthenol, biotin, choline, inositol, and pharmaceutically acceptable salts thereof (e.g., thiamine hydrochloride, thiamine nitrate, dicethiamine hydrochloride, fursultiamine hydrochloride, riboflavin butyrate, riboflavin sodium phosphate, flavin-adenine dinucleotide sodium, pyridoxine hydrochloride, pyridoxal phosphate, pyridoxal calcium phosphate, hydroxocobalamin hydrochloride, hydroxocobalamin acetate, calcium pantothenate, and sodium pantothenate); vitamin C group, for example, ascorbic acid, erythorbic acid, and derivatives or pharmaceutically acceptable salts thereof (e.g., sodium ascorbate and sodium erythorbate); vitamin D group, for example, ergocalciferol, cholecalciferol, hydroxycholecalciferol, dihydroxycholecalciferol, dihydrotachysterol, and pharmaceutically acceptable salts thereof; vitamin E group, for example, tocopherol and derivatives thereof, ubiquinone derivatives and pharmaceutically acceptable salts thereof (e.g., tocopherol acetate, tocopherol nicotinate, tocopherol succinate, and tocopherol calcium succinate); and other vitamins such as hesperidin, carnitine, ferulic acid, γ-orizanol, orotic acid, rutin, eriocitrin, and pharmaceutically acceptable salts thereof (e.g., carnitine chloride).

Examples of the amino acids include leucine, isoleucine, valine, methionine, threonine, alanine, phenylalanine, tryptophan, lysine, asparagine, aspartic acid, serine, glutamine, glutamic acid, proline, tyrosine, cysteine, histidine, ornithine, hydroxyproline, hydroxylysine, aminoethylsulfonic acid, and pharmaceutically acceptable salts thereof (e.g., an equal proportion mixture of potassium aspartate and magnesium aspartate, and cysteine hydrochloride).

Examples of the herbal medicines include processed garlic, ginseng, coix seed, camomile, cinnamon, kakkon-to, ephedra herb, Nandina domestica fruit, Prunus jamasakura bark, polygala root, glycyrrhiza, apricot kernel, plantago seed, plantago herb, Lycoris radiate bulb, senega, ipecac, fritillaria bulb, gambir, fennel, scutellaria root, trichosanthes seed, oriental bezoar, schisandra fruit, asiasarum root, Aster tataricus, musk, Adenophora polymopha root, ginger, mulberry bark, perilla herb, Panax japonicus rhizome, citrus unshiu peel, ophiopogon tuber, and pinellia tuber.

The content of the additional ingredients mentioned above may be appropriately varied depending on various factors including the desired effect and the age and conditions of a subject, but may be, for example, 0.001 to 80 mass %, preferably 0.001 to 30 mass %, and more preferably 0.001 to 10 mass %, based on the total amount of the gastric acid secretion suppressant, the potassium channel inhibitor, or the drug, quasi-drug, food or drink containing the suppressant or the inhibitor.

The dosage form of the gastric acid secretion suppressant, the potassium channel inhibitor, or the drug, quasi-drug, food or drink containing the suppressant or the inhibitor of the present invention is not particularly limited, and any dosage form that is usually used is available. Examples thereof include solid formulations, semi-solid formulations, and liquid formulations. Preferred examples are solid formulations and liquid formulations (e.g., decoctions and infusions), and most preferred examples are solid formulations.

The pharmaceutical preparations of the present invention may be in the forms of tablets (including uncoated tablets, sugar-coated tablets, fast disintegration oral tablets, fast dissolution oral tablets, chewable tablets, effervescent tablets, troches, drops, and film-coated tablets), pills, granules, fine granules, powders, hard capsules, or soft capsules. More preferred dosage forms are tablets, and particularly preferred dosage forms are fast disintegration oral tablets, fast dissolution oral tablets, and chewable tablets, which can be easily taken without water when symptoms of gastric hyperacidity appear or sugar-coated tablets and film-coated tablets, which can block unpleasant tastes.

In addition to the above-described ingredients, the gastric acid secretion suppressant, the potassium channel inhibitor, and the drug, quasi-drug, food or drink containing the suppressant or the inhibitor of the present invention may appropriately contain any ingredients that can be usually used in drugs, quasi-drugs, food or drink, depending on, for example, the application and the dosage form, as long as the effect of the present invention, pharmaceutical stability, and so on are not impaired.

The ingredients that may be contained are not particularly limited, and examples thereof include carrier ingredients or additives.

Examples of the carrier ingredients or the additives that can be contained in the solid formulations include excipients, disintegrators, binders, lubricants, antioxidants, coating agents, coloring agents, taste masking agents, surfactants, plasticizers, sweetening agents, flavoring agents, disintegration aids, foaming agents, adsorbents, preservatives, wetting agents, and antistatic agents. Examples of the carrier ingredients or the additives that can be contained in the liquid formulations include solvents, pH adjusters, refreshing agents, suspending agents, antifoaming agents, thickening agents, solubilizing agents; and the above-mentioned surfactants, antioxidants, coloring agents, sweetening agents, and flavoring agents; and antiseptic and antibacterial agents, chelating agents, solubilizers or solubilizing agents, stabilizers, fluidizers, emulsifiers, thickeners, buffers, isotonizing agents, and dispersants.

Specific examples of the ingredients that can be appropriately blended are shown below, but are not limited thereto.

Examples of the excipients include sugar alcohols such as D-sorbitol, mannitol, and xylitol, saccharides such as glucose, sucrose, lactose, and fructose, crystalline cellulose, carmellose sodium, croscarmellose sodium, calcium hydrogen phosphate, wheat starch, rice starch, corn starch, potato starch, dextrin, β-cyclodextrin, light anhydrous silicic acid, titanium oxide, magnesium aluminometasilicate, talc, and kaolin.

The excipient is preferably mannitol, croscarmellose sodium, or light anhydrous silicic acid, but is not particularly limited thereto.

Examples of the disintegrators include low substituted hydroxypropylcellulose, carboxymethyl cellulose calcium, croscarmellose sodium, hydroxypropyl starch, and partially pregelatinized starch.

Examples of the binders include cellulose derivatives such as methyl cellulose, ethyl cellulose, hydroxypropyl cellulose, and hydroxypropyl methylcellulose, polyvinyl pyrrolidone, polyvinyl alcohol, acrylic acid-based polymers, gelatin, gum arabic, pullulan, pregelatinized starch, agar, tragacanth, sodium alginate, and propylene glycol alginate.

Examples of the lubricants include stearic acid, magnesium stearate, calcium stearate, polyoxyl stearate, cetanol, talc, hydrogenated oil, sucrose fatty acid ester, dimethylpolysiloxane, beeswax, and white beeswax.

The lubricant is preferably magnesium stearate, but is not particularly limited thereto.

Examples of the antioxidant include dibutylhydroxytoluene (BHT), propyl gallate, butylhydroxyanisole (BHA), tocopherol, and citric acid.

Examples of the coating agent include hydroxypropyl methyl cellulose, hydroxypropyl cellulose, methyl cellulose, ethyl cellulose, hydroxypropyl methyl cellulose phthalate, hydroxypropyl methyl cellulose acetate succinate, carboxymethyl ethyl cellulose, cellulose acetate phthalate, polyvinyl acetal diethylaminoacetate, aminoalkylmethacrylate copolymers, hydroxypropyl methyl cellulose acetate succinate, methacrylic acid copolymers, polyvinyl acetate diethylaminoacetate, and shellac.

Examples of the coloring agent include Food Red No. 2, Food Red No. 3, Food Red No. 102, Food Yellow No. 4, Food Yellow No. 5, Food Blue No. 1, Food Yellow No. 4 metal lake, sodium copper chlorophyllin, riboflavin, turmeric extract, and carotene liquid.

Examples of the taste masking agent include aspartame, ascorbic acid, stevia, menthol, crude glycyrrhiza extract, and simple syrup.

Examples of the surfactant include polyoxyethylene hydrogenated castor oil, glyceryl monostearate, sorbitan monostearate, sorbitan monolaurate, polyoxyethylene polyoxypropylene, polysorbates, sodium lauryl sulfate, macrogols, and sucrose fatty acid ester.

Examples of the plasticizer include triethyl citrate, polyethylene glycol, triacetin, and cetanol.

Examples of the sweetening agent include natural or synthetic sweetening agents, such as sucrose, mannitol, and aspartame.

Examples of the flavoring agent include camphor, borneol, and cinnamaldehyde.

Examples of the solvent include water, ethanol, isopropanol, lauryl alcohol, cetanol, stearyl alcohol, oleyl alcohol, lanolin alcohol, behenyl alcohol, 2-hexyldecanol, isostearyl alcohol, and 2-octyldodecanol.

Examples of the pH adjuster include citric acid, malic acid, sodium hydrogen phosphate, and dipotassium phosphate.

Examples of the suspending agent include kaolin, carmellose sodium, xanthan gum, methyl cellulose, and tragacanth.

Examples of the antifoaming agent include dimethylpolysiloxane and silicon antifoaming agents.

Examples of the thickening agent include xanthan gum, tragacanth, methylcellulose, and dextrin.

Examples of the solubilizing agent include ethanol, sucrose fatty acid ester, and macrogol.

The gastric acid secretion suppressant, the potassium channel inhibitor, and the drug, quasi-drug, food or drink containing the suppressant or the inhibitor of the present invention can be produced by a method commonly used in this technical field without modification or with appropriate modification.

For example, tablets can be prepared by mixing a powdered active ingredient and a pharmaceutically acceptable carrier ingredient (e.g., an excipient) and directly compression-molding the mixture (direct tableting method). Drops may be prepared by pouring the mixture into a mold. Among the solid formulations, powder granules such as granules may be prepared by various granulation methods (e.g., extrusion granulation method, crushing granulation method, dry compression granulation method, fluidized-bed granulation method, rolling granulation method, or high-speed stirring granulation method). Tablets can also be prepared by an appropriate combination of, for example, the granulation method and a tableting method (e.g., wet tableting method) (indirect compression method). Furthermore, capsules can be prepared by filling capsules (soft or hard capsules) with powder granules (e.g., powder or granules) by a common method. The tablets may be coated with sugar or film to prepare sugar- or film-coated tablets. Moreover, the tablets may be in the form of single-layer tablets or layered tablets such as double-layer tablets. The liquid formulations can be prepared by dissolving or dispersing each ingredient in an aqueous medium (e.g., purified water, heat-purified water, or ethanol-containing purified water) serving as a carrier ingredient, optionally followed by heating, filtration, fabric filtration, or sterilization, and filling a prescribed container with the resulting solution or dispersion, followed by, for example, sterilization.

### EXAMPLES

The present invention will be described in detail with reference to several examples below, but is not limited to these examples at all.

### Preparation Example 1

A large-intestinal mucosa specimen was prepared in the following manner.

A mouse was killed by cervical vertebra dislocation. After abdominal incision, the cecum was extracted. The boundary parts of the cecum with the small intestine and the large intestine were cut to extract the cecum. The cecum was cut open with scissors into a sheet. In order to completely remove the contents, the cecum was pinched with tweezers and washed with replacement fluid. Replacement fluid was placed in a petri dish covered with rubber, and the cecum was attached thereon with the serosa side up. The muscular layer was separated using tweezers while constantly aerating the replacement fluid in the petri dish with 95% O₂/5% CO₂ to prepare a specimen composed of the mucosa and the submucosa. This specimen was divided into four pieces for use in the experiment.

### Example 1

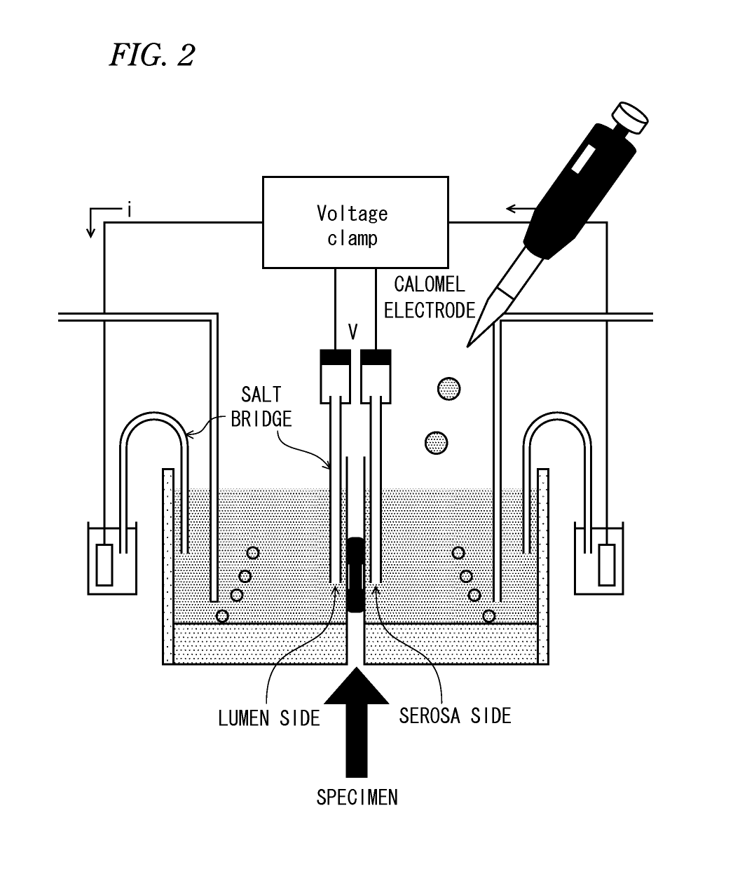

FIG. 2 is an explanatory diagram illustrating a configuration for short-circuit current (Isc) measurement with Ussing chambers.

The mucosa specimen was placed between two Ussing-type chambers each having a window with an area of 0.2 cm² and containing 5 mL of replacement fluid. For the measurement of the electrical potential difference, a pair of calomel electrodes was connected to both chambers through 1 M KCl/2% agar salt bridges. For energization, Ag/AgCl electrodes connected through 1 M NaCl/2% agar salt bridges were installed. These electrodes were connected to a voltage clamp apparatus, and the short-circuit current (Isc) was measured. As for Isc, current flowing from the mucosa side to the serosa side was determined as being positive.

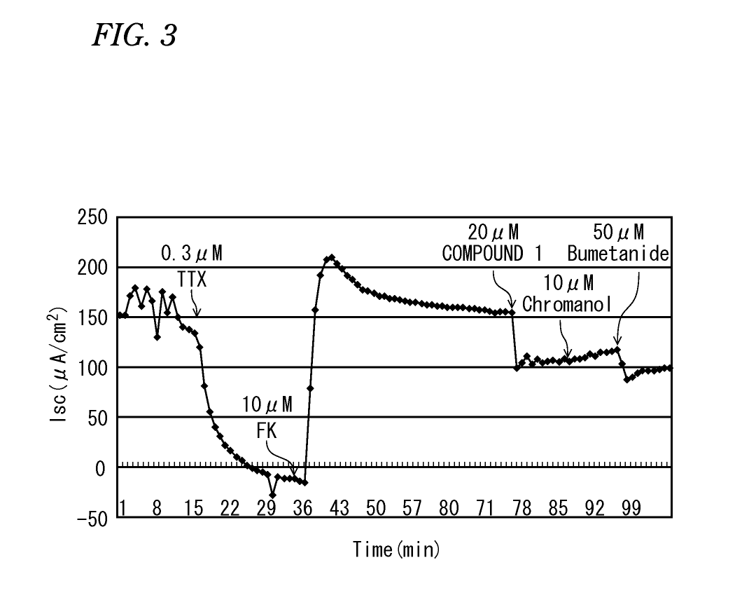

FIG. 3 is an Isc graph showing the effects on Cl⁻ secretion when cooling sensation composition, 2-methyl-3-(menthoxy)propane-1,2-diol (compound 1), and chromanol 293B were administered to the serosa side.

Tetrodotoxin (TTX) was administered to the serosa side to block the nerve, and then forskolin (FK) was administered to the serosa side. By the administration of forskolin, which increases intracellular cAMP level, Isc was significantly increased. At least part of this cAMP-dependent increase in Isc is attributable to the activation of a Cl⁻ secretion mechanism.

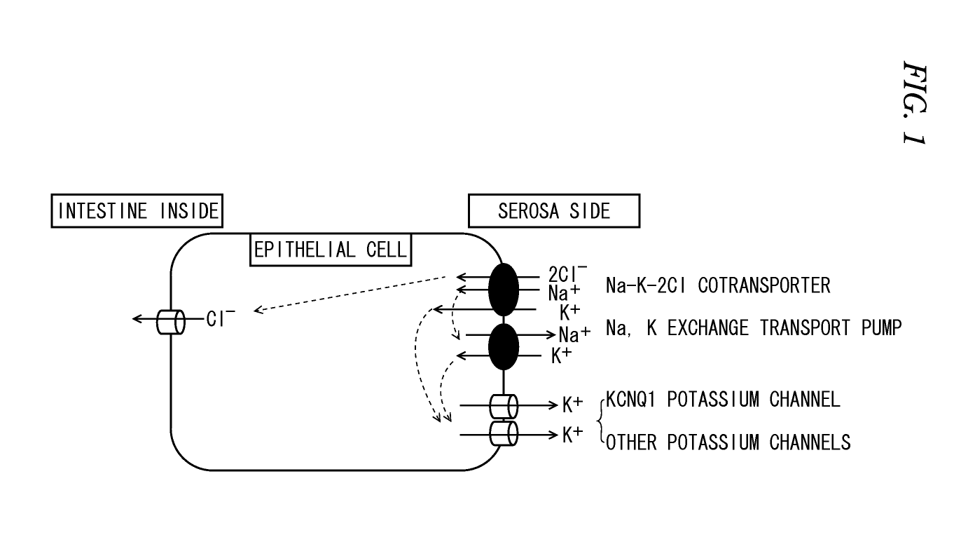

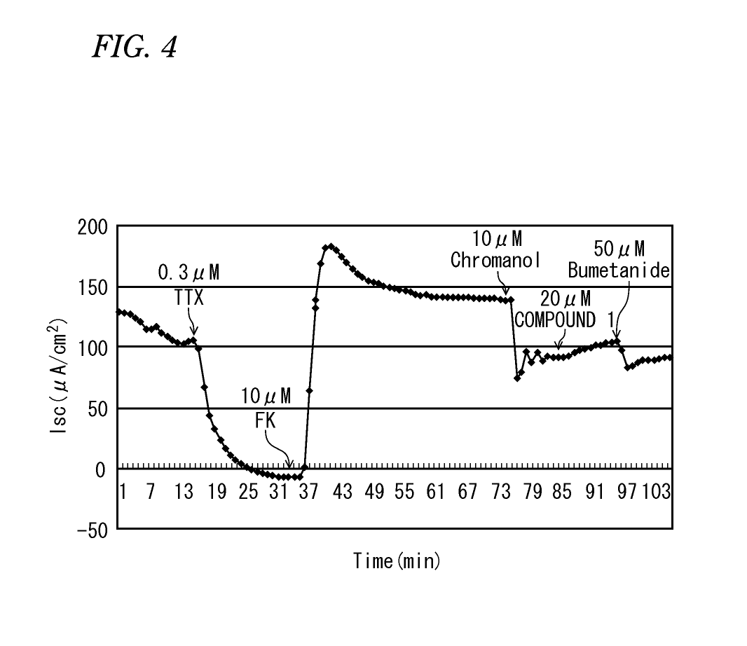

Subsequently, the compound 1 was administered to the serosa side to decrease Isc (to suppress Cl⁻ secretion). Furthermore, chromanol 293B (which inhibits K⁺ channel KCNQ1) was administered to the serosa side, but the Isc-suppressing reaction, which can be observed by administration of chromanol 293B (Example 2 shown in FIG. 4), was not observed. Lastly, a Na⁺.K⁻.2Cl cotransporter inhibitor (which inhibits Cl⁻ secretion) was administered to the serosa side to observe a slight decrease in Isc. Since Cl⁻ secretion is partially supported by K⁺ channels other than KCNQ1 on the serosa side (FIG. 1), even if KCNQ1 is completely inhibited, the Cl⁻ secretion is not completely suppressed, and the slight decrease in Isc is caused by the partially remaining suppression.

### Example 2

FIG. 4 is also an Isc graph similarly showing the effects on Cl⁻ secretion when cooling sensation composition, compound 1, and chromanol 293B were administered to the serosa side.

This was the same experiment as Example 1 shown in FIG. 3 except that chromanol 293B was administered previously. The decrease in Isc due to compound 1 shown in FIG. 3 was not observed when chromanol 293B was administered in advance.

### Example 3

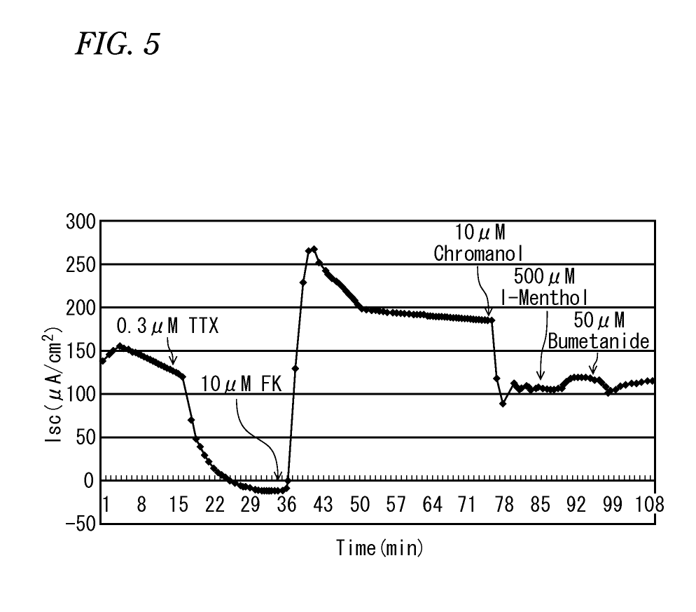

FIG. 5 is also an Isc graph similarly showing the effects on Cl⁻ secretion when cooling sensation composition, menthol, and chromanol 293B were administered to the serosa side.

Also in this case, the administration of menthol did not decrease the Isc when chromanol 293B was administered in advance.

### Preparation Example 2

A stomach specimen was prepared in the following manner.

A mouse was killed by cervical vertebra dislocation. After abdominal incision, the stomach was extracted. The gastric acid secretion portion was cut out, washed with replacement fluid, and divided into two pieces.

Gastric acid secretion activity was measured using this specimen as follows.

### Example 4

### Example 5

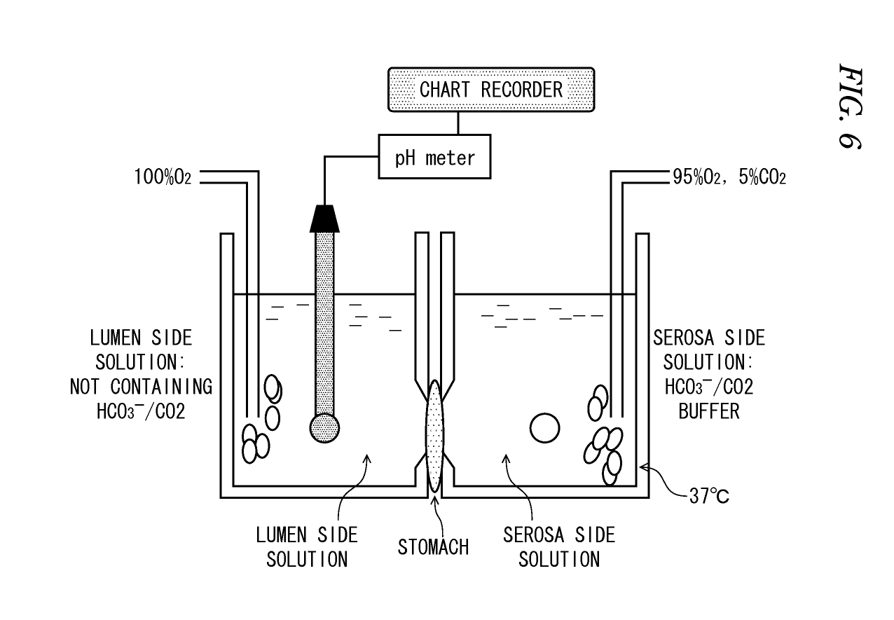

FIG. 6 is an explanatory diagram illustrating a configuration for gastric acid secretion measurement with Ussing chambers.

The specimen was placed between two chambers facing each other and each having a window with an area of 0.2 cm² and containing 10 mL of replacement fluid. Decreases in pH of the replacement fluid in the chamber on the lumen side were successively measured using a pH electrode at 37° C. The gastric acid secretion rate was calculated from the decrease in pH measured after completion of the experiment and the previously measured buffer capacity of the replacement fluid. The experiment was conducted by previously administering histamine (1 mM) to the replacement fluid on the serosa side for stimulating gastric acid secretion.

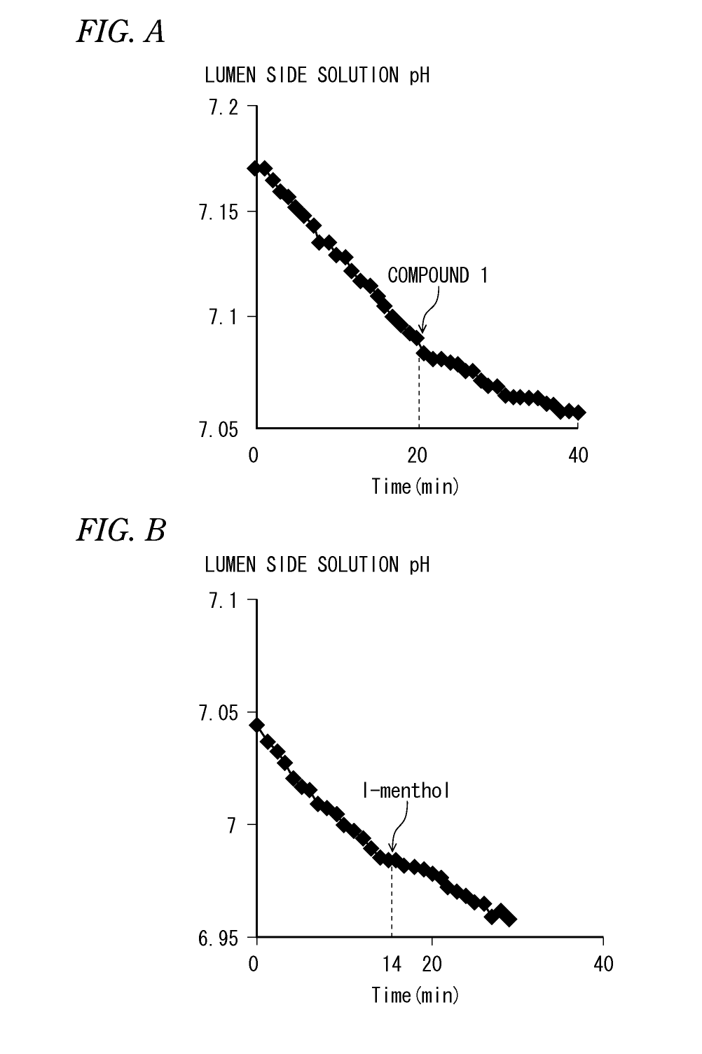

FIG. 7 includes pH graphs (a) and (b) respectively corresponding to Examples 4 and 5 and showing the effects of cooling sensation compositions, compound 1 and 1-menthol, were administered.

L-Menthol and compound 1 were administered to the replacement fluid on the lumen side so that the final concentrations were 50 μM and 100 μM, respectively. The administration was performed using stock solutions each having a concentration of 1000 times that of the target final concentration and using DMSO as a solvent. Compound 1 and 1-menthol were administered at 20 minutes and at 14 minutes, respectively, from the start of the experiment.

The calculated levels of gastric acid secretion rate (micro Eq/cm²/h) are shown in Table 1.

Compound 1 (100 μM) suppressed the gastric acid secretion rate by 75% (Example 1), and 1-menthol did by 57% (Example 2).

TABLE 1

| | Example 4: Gastric acid secretion rate |
|---|---|
| | (micro Eq/cm²/h) |
| Ground state | 1.63 |
| Compound 1 | 0.43 |
| | Example 5: Gastric acid secretion rate |
| | (micro Eq/cm²/h) |
| Ground state | 1.50 |
| l-Menthol | 0.64 |

In addition to the cooling sensation compositions shown in the above-described examples, similar effects can be also obtained by 1-isopulegol, 2-[2-(1-menthoxy)ethoxy]ethan-1-ol, 2-(1-menthoxy)ethan-1-ol, para-menthane-3,8-diol, 1-menthyl 3-hydroxybutanoate, 1-(2-hydroxy-4-methyl-cyclohexyl)-ethanone, N-ethyl-1-menthyl carboximide, 1-menthyl lactate, N-methyl-2,2-isopropylmethyl-3-methylbutanamide, and cooling sensation compositions comparable thereto.

The experimental results shown in the above-described examples are those obtained when L forms were used, but similar effects can be obtained when optical isomers such as D form and DL form are used.

As described above, it was shown that according to the present invention, a potassium channel was inhibited by the action of the cooling sensation composition, and thereby the gastric acid secretion was suppressed.

Several examples of the gastric acid secretion suppressant and the potassium channel inhibitor according to the present invention are shown below, but the present invention is not limited thereto at all.

A prescription example of a gastric acid secretion suppressant is shown in Table 2.

TABLE 2

| Component | Parts by mass (mg) |
|---|---|
| Menthol | 75 |
| 2-Methyl-3-(menthoxy)propane-1,2-diol | 25 |
| Vitamin C | 50 |
| Emulsified oligosaccharide | 90 |
| Granulating agent | 60 |
| Crystalline cellulose | 80 |
| Starch syrup | 90 |
| Sucrose | 60 |
| Flavoring agent | optimum dose |

A prescription example of an antiarrhythmic agent is shown in Table 3.

TABLE 3

| Component | Parts by mass (mg) |
|---|---|
| Rosmarinic acid | 10 |
| Ginsenoside | 10 |
| Menthol | 5 |
| 3-(l-Menthoxy)propane-1,2-diol | 3 |
| Magnesium stearate | 30 |
| Starch | 15 |
| 3% Ethanol solution of polyvidone | optimum dose |

A prescription example of an antianginal agent is shown in Table 4.

TABLE 4

| Component | Parts by mass (mg) |
|---|---|
| Menthol | 20 |
| N-Ethyl-l-menthyl carboxamide | 4 |
| Lactose | 63 |
| Corn starch | 12 |
| Guar gum | 1 |

A prescription example of a peptic esophagitis suppressant is shown in Table 5.

TABLE 5

| Component | Parts by mass (mg) |
|---|---|
| Sodium alginate | 200 |
| Xanthan gum | 35 |
| Locust bean gum | 15 |
| Xylitol | 300 |
| Mannitol | 1225 |
| Povidone K30 | 100 |
| Menthol | 40 |
| 2-(l-Menthoxy)ethan-1-ol | 10 |
| Flavoring agent | 25 |
| Magnesium stearate | 50 |

A prescription example of a gastrointestinal motility disorder suppressant is shown in Table 6.

TABLE 6

| Component | Mass (%) |
|---|---|
| Menthol | 10 |
| 3-(l-Menthoxy)propane-1,2-diol | 15 |
| Lactose | 62 |
| Corn starch | 13 |

A prescription example of a gastrointestinal disorder suppressant is shown in Table 7.

TABLE 7

| Component | Parts by mass (g) |
|---|---|
| Menthol | 1.0 |
| 3-(l-Menthoxy)propane-1,2-diol | 0.5 |
| 2-(l-Menthoxy)ethan-1-ol | 0.5 |
| Nicotinamide | 2.5 |
| Purified water | 96.5 |

A prescription example of an asthma suppressant is shown in Table 8.

TABLE 8

| Component | Mass (%) |
|---|---|
| Menthol | 12 |
| 3-(l-Menthoxy)propane-1,2-diol | 12 |
| Lactose | 63 |
| Corn starch | 12 |
| Guar gum | 1 |

A prescription example of a hypoglycemic agent is shown in Table 9.

TABLE 9

| Component | Parts by mass (mg) |
|---|---|
| Menthol | 50 |
| 3-(l-Menthoxy)propane-1,2-diol | 200 |
| L-Arabinose | 400 |
| Banaba extract powder | 65 |
| Saccharomyces Cr2000 | 40 |
| Zinc yeast | 40 |
| Dextrin | 16 |

### INDUSTRIAL APPLICABILITY

As described above, according to the present invention, the action of the cooling sensation composition effectively contributes to suppression of gastric acid secretion without causing adverse reactions, and the action as a potassium channel inhibitor is effective for improving symptoms and diseases such as arrhythmia, angina pectoris, peptic esophagitis, motility disturbance (including constipation and diarrhea), gastrointestinal disorder (including irritable bowel syndrome), asthma, and hyperglycemia. Thus, the present invention is very useful industrially.

1. A gastric acid secretion suppressant suppressing secretion of gastric acid, comprising:
one or more cooling sensation compositions that are compounds providing cooling sensation.

2. The gastric acid secretion suppressant according to claim 1, wherein
the cooling sensation composition is at least one selected from menthol, menthone, isopulegol, 3-(menthoxy)propane-1,2-diol, 2-(menthoxy)ethan-1-ol, 2-[2-(menthoxy)ethoxy]ethan-1-ol, 3-(menthoxy)propan-1-ol, 2-methyl-3-(menthoxy)propane-1,2-diol, para-menthane-3,8-diol, menthyl 3-hydroxybutanoate, 1-(2-hydroxy-4-methyl-cyclohexyl)-ethanone, N-ethyl menthyl carboxamide, menthyl lactate, and N-methyl-2,2-isopropylmethyl-3-methylbutanamide.

3. The gastric acid secretion suppressant according to claim 2, wherein
the cooling sensation composition has a para-menthane-skeleton and a polar site at the 3-position thereof.

4. A drug, quasi-drug, food or drink comprising a gastric acid secretion suppressant according to any one of claims 1 to 3 as a main ingredient or an accessory ingredient, wherein
the drug, quasi-drug, food or drink is produced by mixing the gastric acid secretion suppressant with another composition.

5. A potassium channel inhibitor inhibiting a potassium channel, comprising:
a cooling sensation composition contained in the gastric acid secretion suppressant according to any one of claims 1 to 3.

6. The potassium channel inhibitor according to claim 5, wherein the cooling sensation composition has an activity effective for improving arrhythmia.

7. The potassium channel inhibitor according to claim 5, wherein the cooling sensation composition has an activity effective for improving angina pectoris.

8. The potassium channel inhibitor according to claim 5, wherein the cooling sensation composition has an activity effective for improving peptic esophagitis.

9. The potassium channel inhibitor according to claim 5, wherein the cooling sensation composition has an activity effective for improving motility disturbance.

10. The potassium channel inhibitor according to claim 5, wherein the cooling sensation composition has an activity effective for improving gastrointestinal disorder.

11. The potassium channel inhibitor according to claim 5, wherein the cooling sensation composition has an activity effective for improving asthma.

12. The potassium channel inhibitor according to claim 5, wherein the cooling sensation composition has an activity effective for improving hyperglycemia.

13. A drug, quasi-drug, food or drink comprising a potassium channel inhibitor according to any one of claims 5 to 12 as a main ingredient or an accessory ingredient, wherein
the drug, quasi-drug, food or drink is produced by mixing the potassium channel inhibitor with another composition.
